(19) United States
(12) Patent Application Publication
(10) Pub. No.: US 2016/0091505 A1
(43) Pub. Date: Mar. 31, 2016

(54) Molecular Template and Manufacturing Method Therefor

(71) Applicant: HITACHI, LTD., Chiyoda-ku, Tokyo (JP)

(72) Inventors: Toshifumi TAKEUCHI, Kobe (JP); Shinichi TANIGUCHI, Tokyo (JP)

(21) Appl. No.: 14/787,844

(22) PCT Filed: Apr. 18, 2014
(86) PCT No.: PCT/JP2014/061051

§ 371 Date: Oct. 29, 2015

(30) Foreign Application Priority Data
May 10, 2013 (JP) 2013-100417

(51) Int. Cl. G01N 33/74

(52) U.S. Cl. CPC G01N 33/743; G01N 2333/575

(57) ABSTRACT

Molecular-template polymer particles for a steroid hormone, said molecular-template polymer particles comprising a polymer that interacts with said steroid hormone. The polymerization unit of said polymer preferably contains at least two functional groups that interact with the aforementioned steroid hormone.

### TECHNICAL FIELD

The present invention relates to a molecular template and a manufacturing method therefor, and a chemical substance detection apparatus and a method for detecting a chemical substance using the molecular template.

### BACKGROUND ART

Chemical substances to be managed in the fields of, for example, clinical laboratory tests, environment, hygiene, and disease control are related to extremely diverse areas, and there are so many different types of such chemical substances. Examples of such chemical substances include: a hormone molecule to serve as a stress disorder marker, endocrine disrupters in the problem of environmental hormones, soil pollution substances in the sites of demolished factories, asbestos released from construction materials, and chemical substances causing malodor or unpleasant taste generated from food and containers thereof, or apparatuses producing food and containers. Most of such chemical substances are a molecule with low molecular weight, and usually contained in extremely small amounts in measurement subjects. However, a rapid and highly sensitive detection of such chemical substances is an extremely important task for the purpose of ensuring the safety or the like in various fields.

Current measurement techniques come to enable the analysis of various chemical substances even at a ppt (one trillionth) or less level as a result of the combination of, for example, highly sophisticated separation techniques, concentration techniques, and analytical methods. The case of such a trace level analysis is usually required to undergo various steps such as optimal separation, concentration, qualitative analysis and quantitative analysis, selected so as to be compatible with the subject for detection. Consequently, such a trace level analysis requires a great deal of labor and a long period of time, and a high analysis cost. Accordingly, an analytical method requiring such a large number of complicated steps is to be specialized as a measurement method in a laboratory, and is not suitable as a method to be used in actual measurement sites.

The measurement method required in actual measurement sites is a measurement method capable of detecting a chemical substance on the site. The sensor technique has developed techniques different from an analysis technique on the basis of such needs. A sensor method enables a simple and rapid detection or monitoring of chemical substances, and additionally is capable of miniaturizing measurement apparatuses.

As the background art of the present technical field, Patent Document 1 and Patent Document 2 can be mentioned. It is described in Patent Document 1 that “devices, methods and kits for rapid and simple qualitative determination of target molecules including small molecules, polypeptides, proteins, cells and infectious disease agents in liquid samples are capable of performing real-time measurement of those entities in liquid samples, and are highly selective, highly sensitive, simple in operability, low in cost and portable. The devices, methods and kits provide, in at least some examples, the use of MIPs in a flow through or lateral flow device” (see Abstract). The MIP as described in this Patent Document 1 means a molecular-template polymer (molecularly imprinted polymer), and the methods for synthesizing MIPs according to the chemical substances to be captured are widely known. There is also a description relating to production of the MIP in Patent Document 2.

### CITATION LIST

### Patent Document

Patent Document 1: WO2009/083975 A2

Patent Document 2: WO2013/046826 A1

### SUMMARY OF THE INVENTION

### Problems to be Solved by the Invention

Unlike the analysis techniques, current sensor techniques have not come to enable the highly sensitive composition analysis of a molecule. However, the measurement usually starts from a state in which the very presence of a chemical substance as a subject to be measured in the aforementioned various fields remains unclear in a specimen. Furthermore, even for a case in which it is present, the amount is generally very small. As such, for the measurement, it is essential to use concentration or separation in combination. However, the measurement method undergoing such process is not different from an analytical method in a scientific laboratory, and it is not suitable as a method to be used in actual measurement sites. Furthermore, as described above, there is a problem that the method cannot be technically dealt with the analytical power of the current sensor techniques.

To solve the aforementioned problems, the inventors of the present invention focused on a molecular-template technique. Namely, provided is a development of a sensor technique for detecting a target chemical substance by selective capturing of a chemical substance without requiring processes like concentration and separation.

An object of the present invention is to provide a molecular-template polymer for capturing a chemical substance in a subject for detection, a method for manufacturing the molecular-template polymer, and a method and an apparatus for detecting, rapidly with high sensitivity at a low cost, a chemical substance allowing identification of the chemical substance by using the molecular-template polymer. Another object of the present invention is to provide a method and an apparatus for detecting a chemical substance, allowing detection of the chemical substance in the subject for detection at an ultrahigh sensitivity.

In other words, the method and apparatus for detecting a chemical substance of the present invention performs the detection by capturing the chemical substance with a capturing body prepared by utilizing a molecular-template polymer. The present invention provides a chemical sensor which is easy to use for general consumers at home as well as for medical personnel (medical doctors, medical technicians, and nurses). In particular, an object of the present invention is to early diagnose the symptom of stress disorder by detecting with high sensitivity a steroid hormone such as cortisol that is closely related to stress disorder, and to herewith contribute to the prevention and early treatment of stress disorder.

### Solutions to Problems

For the purpose of rapidly, inexpensively and highly sensitively detecting a steroid hormone such as cortisol, the present invention solves the foregoing technical problem by synthesizing a molecular-template polymer (MIP) appropriate for a steroid hormone. Specifically, for example, the present invention adopts the constitution described in the claims. The present application includes a plurality of means for solving the foregoing technical problem; as an example to be quoted of such means, the molecular-template polymer according to the present invention is characterized by “the molecular-template polymer for a steroid hormone, in which the molecular-template polymer consists of a polymer that interacts with the steroid hormone.”

Although the synthesis principle of a polymer has been known since 1950s, it is necessary to elaborately investigate the synthetic raw materials, synthetic pathway, reaction time and reaction temperature appropriate for a chemical substance (target) to be captured. Thus, it is possible to propose as a principle the device structure utilizing such a polymer as quoted in foregoing Patent Document 1; however, for the purpose of practically preparing a polymer to capture a target with a high sensitivity, an elaborate design, an elaborate synthesis and an elaborate purification come to be necessary.

Furthermore, there is a description in Patent Document 2 relating to the molecular-template polymer for cortisol, and the method for producing the polymer is a polymerization reaction using cortisol and a raw material monomer.

A molecular-template polymer prepared by molecular imprinting can be constructed by using various matrices. The present inventors have discovered a polymer to be used in molecular imprinting for the molecule of a steroid hormone such as cortisol or the derivatives of cortisol closely related to stress disorder.

In the present invention, the polymerization reaction is performed by using particles as a core, modified cortisol, and a raw material monomer. Accordingly, the invention is characterized in that the molecular-template polymer with highly fine spherical shape is produced.

The molecular-template polymer in the present invention is appropriate as the matrix to be used for imprinting of a steroid hormone in that the network structure of the polymer has an appropriate flexibility, and is swollen or shrunken according to a factor such as a solvent or an environment. In other words, the recognition site formed by the template molecule in the molecular-template polymer is required to be close in size to the template molecule. On the other hand, for the purpose of removing the template molecule after the polymerization or allowing the chemical substance (target) to bind again to the recognition site, a space large to some extent is required so as to allow the molecule to migrate in the network structure. The present inventors have discovered a polymer material satisfying such conflicting conditions and the synthesis conditions of the polymer material. In particular, a steroid hormone such as cortisol has a steroid skeleton, and hence the molecule thereof is rigid. Further, since a steroid hormone such as cortisol has groups such as hydroxyl groups, it is capable of forming interactions with the raw material monomer, required at the time of molecular imprinting. In particular, in the present invention, by using a dicarboxylic acid derivative capable of interacting with, for example, cortisol at two sites of a part of the raw material monomer, a synthesis of a molecular-template polymer enabling a highly efficient capture is achieved.

In addition, according to the present invention, a methacroylated cortisol derivative for having a polymerizable substituent group according to modification of a part of a cortisol molecule is used instead of cortisol for synthesis of the molecular-template polymer. As a result, a covalent bond to the monomer molecule as a raw material of a molecular-template polymer is formed, and thus synthesis of a molecular-template polymer enabling highly efficient capturing is achieved.

Thus, the method for detecting a chemical substance of the present invention acquires a highly sensitive detection capability by constituting the method to enhance the sensitivity of detection of the captured steroid hormone.

### Effects of the Invention

According to the method and apparatus for detecting a chemical substance of the present invention, it is possible to selectively detect a steroid hormone to be detected owing to the molecular-template polymer formed of a specific polymer, without needing any concentration step or any separation step.

Furthermore, according to the apparatus for detecting a chemical substance of the present invention, it is possible to reduce the size of the molecule capturing part corresponding to the most important sensor portion, and hence it is possible to provide a portable apparatus for detecting a chemical substance.

### BRIEF DESCRIPTION OF THE DRAWINGS

FIG. 1 is a diagram schematically illustrating a representative method for manufacturing a molecular-template polymer.

FIG. 2 is a longitudinal cross-sectional view for describing the concept of an apparatus for detecting a chemical substance according to a first embodiment of the present invention.

FIG. 3A is a diagram illustrating a synthesis scheme of the molecular-template polymer according to the first embodiment.

FIG. 3B is a diagram schematically illustrating a method for manufacturing molecular-template polymer particles.

FIG. 4A is a diagram schematically illustrating the molecular structure of cortisol.

FIG. 4B is a diagram schematically illustrating the molecular structure of itaconic acid.

FIG. 5 is a diagram schematically illustrating the interaction between cortisol and itaconic acid.

FIG. 6 is a diagram schematically illustrating the molecular structure of various steroid hormones.

FIG. 7 is a conceptual diagram for describing a competition method according to a second embodiment of the present invention.

FIG. 8 is a perspective view illustrating an embodiment of an apparatus for detecting a chemical substance according to a third embodiment of the present invention.

FIG. 9 is a diagram illustrating the molecular structure of methacroylated cortisol according to Example 1.

FIG. 10A is a diagram illustrating the molecular structure of the cortisol derivative according to Example 2.

FIG. 10B is a diagram illustrating the molecular structure of the cortisol derivative according to Example 2.

FIG. 11A is a graph showing the detection results of cortisol of Example 2.

FIG. 11B is a graph showing the detection results of cortisol of Example 2.

FIG. 12 is a graph showing the detection results of cortisol of Example 3.

FIG. 13A is a diagram showing the molecular structure according to Example 4.

FIG. 13B is a diagram showing the molecular structure of the cortisol derivative according to Example 4.

### MODE FOR CARRYING OUT THE INVENTION

Hereinafter, the embodiments of the present invention are described. The present invention is not limited to these embodiments at all, and can be implemented in various modes within a range not departing from the gist of the present invention.

An embodiment of the apparatus for detecting a chemical substance of the present invention is constituted with a molecule capturing part having on the surface thereof a capturing body including a molecular-template polymer that is formed by utilizing a specific chemical substance, and a part for measuring captured amount for quantitatively determining the chemical substance captured in the molecule capturing part. The capturing body can capture the specific chemical substance (target) in a specimen in a manner depending on the specific molecular structure possessed by the chemical substance. The apparatus for detecting a chemical substance of the present embodiment performs molecular recognition of a chemical substance on the basis of this technique.

FIG. 1 illustrates the representative preparation principle of a molecular-template polymer 22, which is applied to the present embodiment. First, a polymerization reaction is performed in a mixture composed of a target 20 to be captured, and a monomer raw material A 201, a monomer raw material B 202 and a monomer raw material C 203, which interact with the target 20, and thus a recognition site 21 of the target 20 is formed. Subsequently, the target 20 is removed by an operation such as washing, and thus the molecular-template polymer (MIP) 22 having a recognition site 21 can be prepared. Here is shown an example in which the target 20 is used as the template molecule for forming the recognition site 21; however, in place of the target 20, the derivatives and analogs of the target 20 may also be used.

### FIRST EMBODIMENT

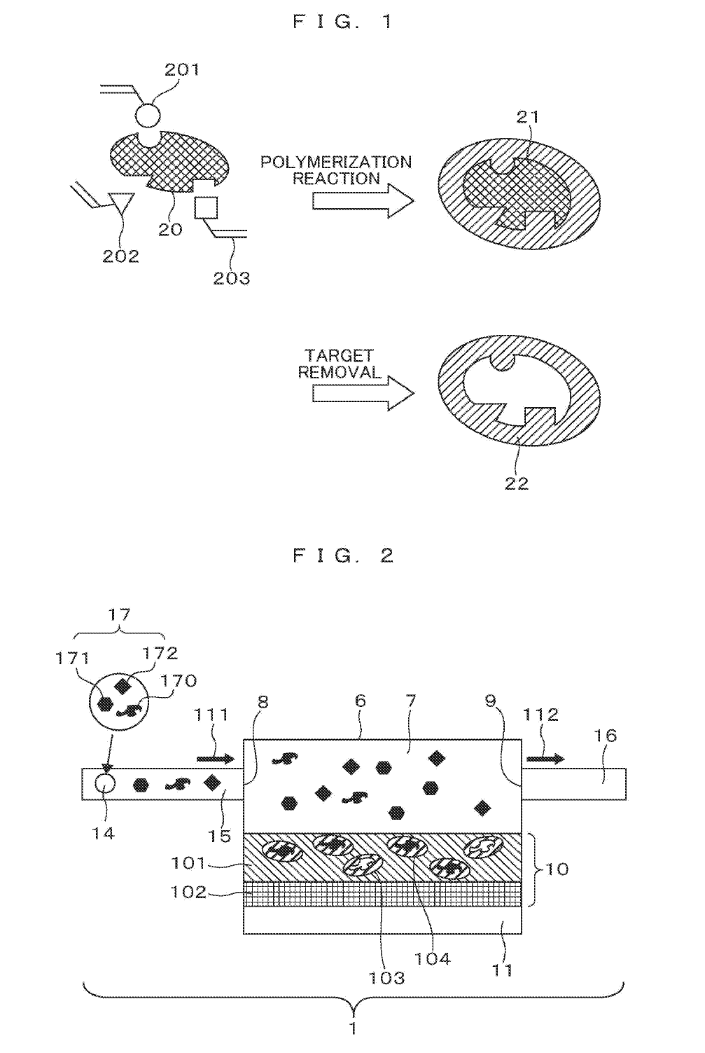

FIG. 2 is a longitudinal cross-sectional view for describing the concept of an apparatus for detecting a chemical substance according to a first embodiment of the present invention. As illustrated in FIG. 2, an apparatus 1 for detecting a chemical substance according to the present embodiment includes one or more sample chamber 6, a sample injection part 14, a sample conveying part 15, and a discharge part 16. The sample chamber 6 has a liquid flow path part 7, a detachable part for connecting the liquid flow path part 7 to the sample conveying part 15 via a flow injection port 8 and a flow discharge port 9, a molecule capturing part 10 present below the liquid flow path part 7 for communication with the liquid flow path part 7, and a part for measuring captured amount 11. When plural sample chamber 6 are installed in the apparatus 1 for detecting a chemical substance, there can be a structure in which the sample conveying part 15 is branched out into several parts. Furthermore, for each of the sample chamber 6 to be connected to the individually branched sample conveying part 15, each detachable part is provided with a valve corresponding to the flow injection port 8 and the flow discharge port 9. Hereinbelow, each constitution is described in detail.

Into the sample injection part 14, a specimen 17 is injected. The specimen 17 includes a target 170, which is a subject for detection, a foreign substance A 171 and a foreign substance B 172. Needless to say, some specimens include no target 170, or a larger number of types of foreign substances. The specimen 17 is conveyed in the direction of an arrow 111 or an arrow 112.

The molecule capturing part 10 is constituted with a capturing body 101 and a support 102. The capturing body 101 includes a molecular-template polymer 103 before capturing the target or a molecular-template polymer 104 after capturing the target. The capturing body 101 is disposed on the surface of the molecule capturing part 10, and is mainly composed of a molecular-template polymer (MIP). The support 102 supports the capturing body 101, and it is a solid constituting the main shape of the molecule capturing part 10. The material of the support 102 is not particularly limited, as long as the material can maintain a predetermined shape. Specific examples of the material of the support 102 include: plastic, metal, glass, synthetic rubber, ceramic, water-proofed or reinforced paper, or combinations of these. In the molecule capturing part 10, the surface having the capturing body 101 may be a surface covering the whole or a portion of the molecule capturing part 10.

The molecule capturing part 10 can be formed by combining the separately and independently prepared capturing body 101 and support 102. The support 102 may be constituted with a multilayer structure composed of different constituent components. For example, there can be a case in which the support 102 is composed of two layers, namely, a glass substrate and a gold (Au) thin film. The method for combining the capturing body 101 and the support 102 is not particularly limited, as long as the molecule capturing part 10 is constituted in such a way that the target capturing information can be delivered to the below-described part for measuring captured amount 11. For example, the capturing body 101 and the support 102 may be directly combined with each other, or may be combined through one or more other connection substances to connect these two members. The molecule capturing part 10 may also be constituted by integrating the capturing body 101 and support 102 made of the same material. For example, there can be a case in which a polymer itself including a molecular-template polymer also plays the role of a support.

The molecule capturing part 10 is constituted at least in such a way that the surface having the capturing body 101 can be directly brought into contact with the specimen 17, so as for the capturing body 101 to capture the target 170 to be detected. As described herein, the “specimen” means a liquid or a solid to be a measurement subject.

The capturing means the capturing through bonding or interaction. The capturing is a concept including both direct capturing and indirect capturing. For example, the capturing may be a direct capturing of the target 170 to be detected, by the capturing body 101 of the molecule capturing part 10, or an indirect capturing of the target to be detected, through a second capturing body fixed on the molecule capturing part.

The capturing body 101 includes the molecular-template polymer formed by using a specific template molecule, and can capture the chemical substance as a target in a manner depending on the specific molecular structure possessed by the target. The material of the capturing body 101 is not particularly limited as long as the material has a function to capture the target in a manner depending on the specific molecular structure. For example, the material may be a protein, a polymer or a metal. Specifically, for example, there are antibodies and molecular-template polymers.

The method for manufacturing the molecular-template polymer used in the present invention is as follows. For example, first, in the presence of a target or a target-like chemical substance, a functional monomer that interacts with the target or the target-like chemical substance through ionic bond or hydrogen bond is copolymerized with other monomer components used if necessary, and thus, the target or the target-like chemical substance is fixed in the polymer. In this case, the copolymerization ratio between the functional monomer and the other monomer components is varied depending on, for example, the types of the individual monomer components and not particularly limited; however, for example, it is possible to set such that the functional monomer:other monomer components=1:16 to 1:64 (molar ratio). In particular, it is desirable that this ratio be 1:32. Subsequently, the target is removed from the polymer by washing. The cavity (space) left in the polymer memorizes the shape of the target, and is provided with a chemical recognition ability due to the functional monomer fixed in the cavity.

In the present embodiment, as the target, an example of a steroid hormone is described; however, examples of the target include, without being limited to steroid hormones, various substances being present, under normal temperature and normal pressure, in a state of being vaporized or in a state of being liquid (inclusive of, for example, the case of being dissolved in a solvent). For example, volatile chemical substances, electrolytes, acids, bases, carbohydrates, lipids and proteins are included. Examples of the target also include the chemical substances capable of being present only in a state of being a solid under normal temperature and normal pressure, and capable of being present as particles in gases or in liquids. The targets which have corrosion effects, dissolution effects, modification effects and the like on the molecule capturing part are not suitable.

The molecular weight of the target is not particularly limited as long as the molecular weight allows the capturing body 101 to capture the target; however, in the present invention taking as its main object the detection of chemical substances with low molecular weight, the target is preferably a molecule with low molecular weight having a molecular weight of the order of a few tens to a few hundreds.

The molecule capturing part 10 may be constituted in a manner detachable from the apparatus 1 for detecting a chemical substance with the aid of a detachable part. This is for the purpose of enabling the selection of an optimal molecule capturing part in plural molecule capturing parts 10 according to, for example, the measurement environment or the state of the specimen, or for the purpose of omitting the labor and time for washing a once used molecule capturing part, and additionally, for the purpose of precluding the risk of contamination due to continuous use. The molecule capturing part detached with the aid of the detachable part is not necessarily required to be the whole of the molecule capturing part, but, for example, the sample chamber 6 is provided with plural capturing parts 101 and only some of them may be detached. Furthermore, it is also possible that each of the molecule capturing part 10 and the part for measuring captured amount 11 of the sample chamber 6 is formed so as to form a pair, or one or plural molecule capturing parts, or one or plural parts for measuring captured amount are independently provided and their combination is arbitrarily modified to allow the optimum measurement.

The detachable part may have, for example, a fixing member for fixing the molecule capturing part 10 to the apparatus 1 for detecting a chemical substance, and a terminal for transmitting information to and receiving information from the molecule capturing part 10. In a case in which an apparatus 1 for detecting a chemical substance has a plurality of molecule capturing parts 10, there may be a plurality of detachable parts.

The part for measuring captured amount 11 is constituted so as to be capable of quantitatively determining the chemical substance captured in the molecule capturing part 10. It is provided with a metal thin film for measurement, for example. The phrase of the “quantitative determination of a chemical substance” means the measurement of the amount of the molecules of the chemical substance to be the target, captured by the capturing body 101 when the specimen 17 is exposed to the molecule capturing part 10 for a predetermined period of time. The “predetermined period of time” as referred to herein means an optional period of time determined in advance before the quantitative determination. For example, the predetermined period of time may be one second or one minute. In the quantitative determination, the dynamic change of the capturing body 101, when the capturing body 101 captures the target 170 present in the specimen 17, is transformed into an electric signal, and on the basis of, for example, the intensity of the electric signal, the captured target can be measured. The method for the quantitative determination is not particularly limited as long as the dynamic change of the capturing body 101 can be transformed into an electric signal. For example, a surface plasmon resonance measurement method, a quartz crystal microbalance measurement method, an electrochemical impedance method, a colorimetric method or a fluorescence method can be adopted. The quantitative determinations based on these methods can all perform the measurement in a period of time of 100 ms (0.1 second) or less.

The surface plasmon resonance measurement method is also referred to as the SPR (surface plasmon resonance) method, and is a method for measuring with a high sensitivity a trace amount of a captured subject on a metal thin film by utilizing the surface plasmon resonance phenomenon such that with the variation of the incidence angle of a laser light beam incident on the metal thin film, the reflected light intensity is attenuated. Specifically, the molecular-template polymer of the present invention is suspended in a solvent (water, or an organic solvent), and the capturing body 101 is spin-coated on a metal thin film of the support 102 in the sample chamber 6, dried and subjected to a measurement. In the measurement, the surface plasmon is generated on the surface side of the metal thin film. When the wave number of an evanescent wave and the wave number of a surface plasmon coincide with each other, the photon energy is used for exciting the surface plasmon through resonance, and hence a phenomenon of the attenuation of the reflected light is caused. This can be found as the attenuation of the reflected light intensity accompanying the variation of the incidence angle of the laser when the incidence angle of the laser is varied. The incidence angle (referred to as the resonance angle θ) when the reflected light intensity ratio, which is the ratio of the reflected light intensity to the incident light intensity, becomes minimum is affected by the interaction between substances occurring on the metal surface. Accordingly, the interaction between the substances can be observed as the variation of the resonance angle θ between before and after the interaction. For example, when the resonance angle in the state in which the molecular-template polymer supported on the metal thin film surface of the support 102 does not capture anything is represented by θ₀, the resonance angle is changed to θ₁ when the molecular-template polymer captures the target. In this case, by observing the value of Δθ as the difference between θ₁ and θ₀, the amount of the target captured by the molecular template can be quantitatively determined. Accordingly, for example, cortisol contained in the specimen in a concentration of 125 μM can be quantitatively determined. When spin coating is performed, it is important to form the film of the capturing body 101 within the distance over which the plasmon resonance propagates. Specifically, it is preferable to form the film of the capturing body 101 with a thickness of 100 nm or less.

The quartz crystal microbalance measurement method is also referred to as the QCM (quartz crystal microbalance) method, and is a mass measurement method of quantitative determination of an ultra-trace amount of a substance attached on a quartz crystal on the basis of the variation magnitude of the resonance frequency of the quartz crystal due to the attachment of the substance on the surface of the quartz crystal. Specifically, the molecular-template polymer of the present invention is suspended in a solvent (water, or an organic solvent), and the capturing body 101 is spin-coated on the sensor of a quartz crystal, dried and subjected to a measurement. The measurement method is a heretofore known established method, thus the measurement may be performed according to the conventional technique, and hence a detailed description of the measurement is omitted. For the purpose of achieving the measurement reproducibility, the thickness of the film of the capturing body 101 formed on the quartz crystal is preferably set at 1 μm or less.

The electrochemical impedance method is also referred to as the surface-polarization controlling method, and is a method in which by controlling the surface polarization of a metal through the electrode potential, the interaction between the electrode surface and the substance attached to the electrode surface is varied, and thus the information about the attached substance is obtained. Specifically, the molecular-template polymer particles of the present invention are suspended in a solvent (water, or an organic solvent), the capturing body 101 is spin-coated on the electrode surface, dried and subjected to a measurement. The measurement method is a heretofore known established method, thus the measurement may be performed according to the conventional technique, and hence a detailed description of the measurement is omitted. For the purpose of achieving the measurement reproducibility, the thickness of the film of the capturing body 101 formed on the quartz crystal is preferably set at 1 μm or less.

The colorimetric method and the fluorescence method are different from each other only in the nature of the substrate used for detection, and are almost the same as each other in the principle involved. Specifically, in a case in which the substrate produces a chromogenic substance, the method concerned is referred to as the colorimetric method; in a case in which the substrate produces a fluorescent substance, the method concerned is referred to as the fluorescence method. In either of these methods, the substrate or the like as the probe for detection is supported on the capturing body, or on a mediator or the like, the color concentration or the fluorescence intensity based on the substrate is measured with an absorption spectrophotometer or a luminometer or the like, and thus, the binding with the target is quantitatively determined.

For a case in which the capturing body is an antibody, these methods correspond to the ELISA method and the like. The ELISA method is also referred to as the enzyme linked immunosorbent assay method. The principle of the ELISA method is such that the primary antibody bound to the target is made to produce a chromogenic substance or a fluorescent substance by the action of the enzyme concerned, for example, through the secondary antibody, which is an enzyme-labeled mediator, and the target is quantitatively determined on the basis of the color concentration of the chromogenic substance or the fluorescence intensity of the fluorescent substance.

In the case of the molecular-template polymer, for example, there can be a molecular-template polymer having in the cavity a functional monomer supporting a substrate probe. For example, the capturing of the target in the molecular-template polymer changes the state of the substrate probe in the cavity so as to develop a color or emit fluorescence, and the target can be quantitatively determined on the basis of the color concentration of the developed color or the fluorescence intensity of the emitted fluorescence.

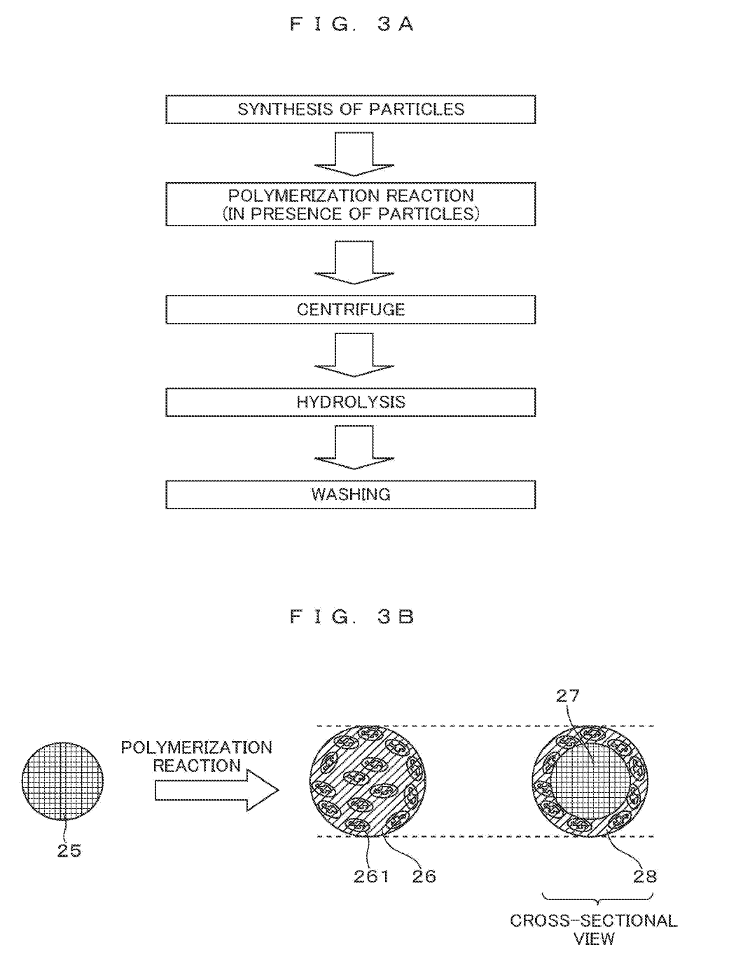

FIG. 3A illustrates a synthesis scheme of the molecular-template polymer particles. The present invention is characterized in that the particles are synthesized first, and in the presence of the synthesized particles, a target and a polymerizable vinyl monomer are subjected to a polymerization reaction to produce the particles of a molecular-template polymer. After that, by performing the centrifuge process, hydrolysis process, and washing process, particles of a molecular-template polymer can be obtained. FIG. 3B is diagram schematically illustrating the process for manufacturing a synthesized molecular-template polymer particles based on that synthetic scheme. A particle 25 with fine spherical shape is coated by a molecular-template polymer 26 via a target or a target derivative which becomes the template and the raw material (monomer) of the molecular-template polymer. The coated molecular-template polymer with fine spherical shape has a target recognition site 261. When the cross-sectional view of the particles coated by the molecular-template polymer 26 with fine spherical shape is shown, it is evident that there are particles 27 and a molecular-template polymer 28 coating the particles 27. As it is a particle with two-layer structure having a nucleus (core), the molecular-template polymer particles of the present invention constitute a core and shell type.

The obtained molecular-template polymer particles have a submicron size and even particle diameter. Thus, when the molecular-template polymer particles are arranged on top of a column or a flat plate, a densely filled state is provided, and thus the target recognition capability is high.

Synthesis of molecular-template polymer particles for cortisol, which is one type of a steroid hormone, according to the order described above is described hereinbelow. Meanwhile, the method described below represent a method for manufacturing a molecular-template polymer which has higher recognition power for cortisol, according to forming of a covalent bond between cortisol and part of the molecular-template polymer particles surrounding the cortisol. However, as long as a molecular-template polymer is produced in the presence of particles, the interaction between cortisol and surrounding vinyl monomer is not limited to a covalent bond, and part or combination of an ionic bond, a hydrogen bond, van der Waals force, and a hydrophobic-hydrophobic bond can be utilized.

### (Manufacturing of Molecular-Template Polymer Particles for Cortisol)

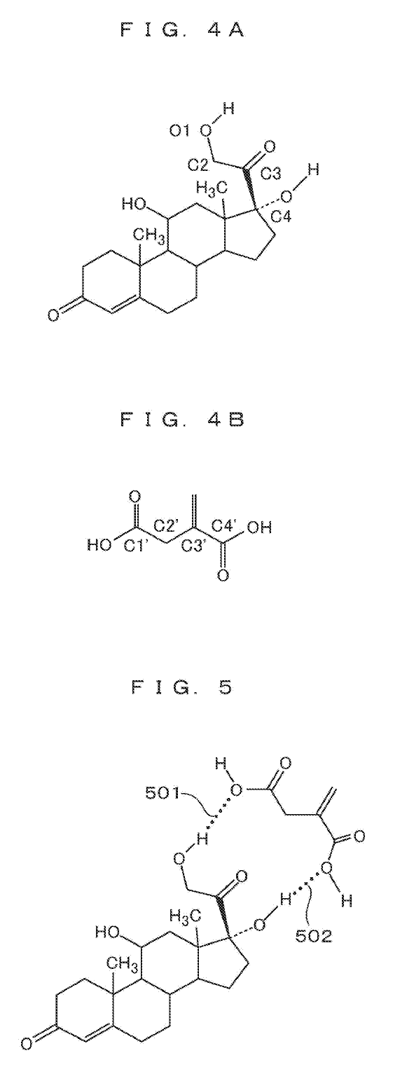

In the presence of particles as a core and cortisol as target, the polymerization of a functional monomer interacting with cortisol monomer is performed, and by washing the polymer obtained by the polymerization reaction, it is possible to obtain a molecular template, specifically recognizing cortisol, in the interior of the polymer. FIG. 4A illustrates the molecular structure of cortisol. FIG. 4B illustrates the molecular structure of itaconic acid. As illustrated in FIG. 4A, when the carbon atom in the terminal five-membered ring in the skeleton of cortisol is denoted by C4, the carbon atom next to C4 of the carbonyl group can be denoted by C3, the carbon atom next to C3 of the methylene group can be denoted by C2, and the oxygen atom next to C2 of the hydroxyl group can be denoted by 01. When the skeleton is assumed to extend to 01, the bonds are formed from the terminal of the steroid skeleton, through two carbon atoms, to the oxygen atom.

Meanwhile, in itaconic acid as illustrated in FIG. 4B, when the carbon atom of the carboxyl group on the left side of the figure is denoted by C1′, the carbon atom next to C1′ of the methylene group can be denoted by C2′, the carbon atom next to C2′ of the vinyl group can be denoted by C3′, and the carbon atom next to C3′ of the carboxyl group can be denoted by C4′.

The important point in the manufacturing of the molecular-template polymer of the present invention is the strength of the interaction force between the target and the polymerizable monomer. The use of itaconic acid as the raw material for the molecular-template polymer of cortisol is based on the reason that carboxyl groups are located on both terminals of the itaconic acid molecule, the distance between these groups is appropriate, and accordingly, itaconic acid may easily interact with a moiety of cortisol. FIG. 5 schematically illustrates the interaction between cortisol and itaconic acid. As the dotted line 501 and the dotted line 502 indicate, the use of itaconic acid allows the interaction with cortisol to occur at a plurality of sites. In this way, the inclusion, in the polymerization units, of a monomer having two or more functional groups that interacts with a steroid hormone such as cortisol improves the fitting property between the steroid hormone and the monomer, and thus a significant property as the molecular-template polymer is considered to be able to be brought about.

The “functional group” as referred to herein means an atomic group included in common in a group of chemical substances and exhibiting common chemical properties and reactivities in the group of chemical substances. Examples of the functional group include: a hydroxyl group, an aldehyde group, a carboxyl group, a carbonyl group, a nitro group, an amino group, a sulfone group and an azo group. When the monomer that interacts with a steroid hormone has two or more functional groups, the functional groups are particularly preferably carbonyl groups.

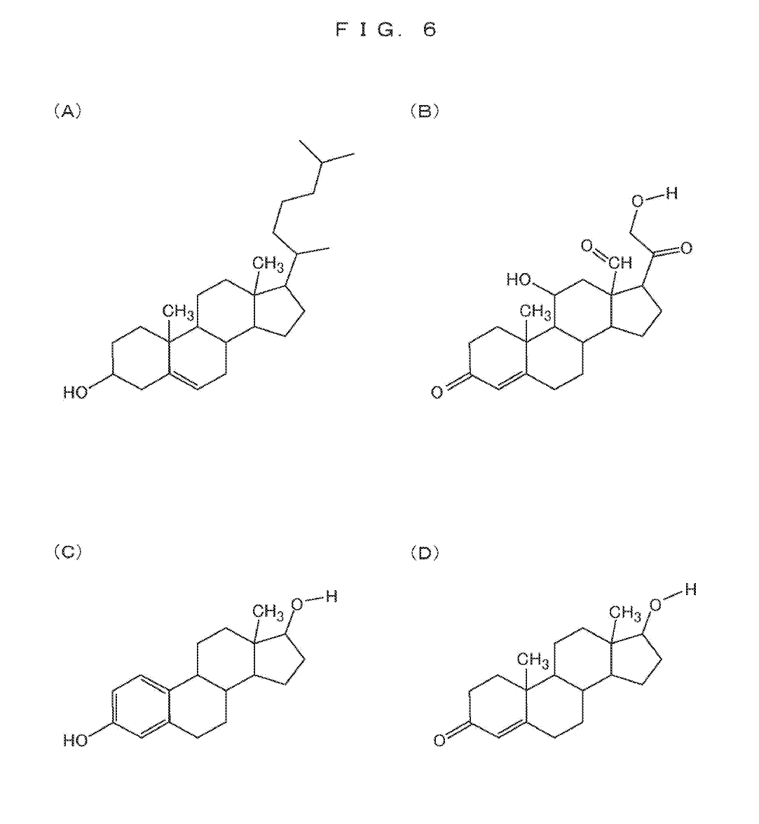

For steroid hormones other than cortisol, by utilizing polymerizable monomers to interact preferably at a plurality of sites, molecular-template polymers can be synthesized. Natural steroid hormones are generally synthesized from cholesterol in gonads and adrenal glands. FIG. 6 illustrates the molecular structures of cholesterol and representative steroid hormones. When the foregoing method for synthesizing the molecular-template polymer particles for cortisol is used, molecular-template polymer particles for other steroid hormones can be prepared. (A) of FIG. 6 illustrates cholesterol, and aldosterone of (B) of FIG. 6, estradiol of (C) of FIG. 6 and testosterone of (D) of FIG. 6 are metabolically synthesized with the cholesterol as mother skeleton.

As described above, itaconic acid is preferably used as the raw material for the molecular-template polymer for cortisol, as a result of selecting the raw material aiming at formation of hydrogen bonds at multiple sites. Similarly to this, it is possible to select a monomer structure suitable for a steroid hormone having a steroid skeleton high in planarity. For aldosterone of (B) of FIG. 6, a monomer raw material for a molecular-template polymer can be selected by paying attention to the two items, namely, the hydroxyl group (OH) present at a terminal through the intermediary of a carbonyl group and a methylene group, and the aldehyde group (CHO) directly bonded to the skeleton. It is recommended to use, as a raw material for the molecular-template polymer, a monomer molecule having a length allowing simultaneously interacting with such a plurality of functional groups as described above. Specifically, it is recommended to manufacture a molecular-template polymer by using as a polymerization unit a monomer which is a vinyl monomer, has two carboxyl groups in the skeleton thereof, and has a distance (a distance of two or three in terms of methylene group) appropriate for fitting to the target of the molecular-template polymer. Similarly to the molecular-template polymer of cortisol, in the presence of the steroid hormone to be the target, the foregoing vinyl monomer and other monomer components such as styrene and divinylbenzene are copolymerized if necessary together with a polymerization initiator, and thus, a molecular-template polymer can be obtained. Instead of performing copolymerization, the vinyl monomer to undergo the interaction may also be homopolymerized. When the foregoing vinyl monomer and other monomer components are copolymerized with each other, the copolymerization ratio between these monomers is varied depending on the individual monomer components and the type and the like of the steroid hormone, and is not particularly limited; however, the copolymerization ratio between these monomers can be specified for example as follows: vinyl monomer that interacts with steroid hormone:other monomer components=1:16 to 1:64 (molar ratio). The foregoing ratio is particularly preferably 1:32.

Estradiol of (C) of FIG. 6 and testosterone of (D) of FIG. 6 each have functional groups distant from each other, and accordingly at the time of producing a molecular-template polymer, one monomer is not required to simultaneously interact at a plurality of sites, and it is sufficient to perform copolymerization, by using a plurality of polymerizable monomers respectively recognizing the functional groups, in the presence of the target, together with styrene, divinylbenzene, a polymerization initiator and the like.

In the foregoing example, a description is made on the case in which the interaction due to hydrogen bonding is formed between a steroid hormone and a monomer; however, as another embodiment, the steroid hormone to be a template molecule is converted into a derivative, and into the derivative, functional groups to undergo copolymerization reaction with the monomer to form the molecular-template polymer may also be introduced. The formation of the covalent bonds between the steroid hormone and the monomer due to the copolymerization reaction may yield a stronger interaction between the steroid hormone and the monomer, improves the fitting property between the steroid hormone and the monomer, and can provide the advantageous properties as the molecular-template polymer. As the monomer to be copolymerized with the steroid hormone, similarly to the foregoing, monomers having two or more functional groups such as itaconic acid and a plurality of types of monomers can be used in combination.

Furthermore, examples of the functional groups to be introduced into the steroid hormone molecule and to be copolymerized with the monomer include polymerizable substituent groups such as an acryloyl group, a methacryloyl group, a vinyl group and an epoxy group. Among these, in particular, a methacryloyl group is preferable.

### SECOND EMBODIMENT

As a second embodiment of the present invention, the molecule capturing part of the apparatus for detecting a chemical substance may be constituted so as to enhance the sensitivity of detection of a steroid hormone with the aid of the competition method or the substitution method.

The “substitution method” is a method utilizing the competition with respect to the capturing body, occurring between a chemical substance having a specific molecular structure captured in advance by the capturing body and the target to be detected in the specimen. For example, when the capturing body is an antibody, the antibody is fixed on a support, and a composite antigen having a specific molecular structure is captured in advance by the antibody. In this state, when the specimen including the target to be detected is exposed to the molecule capturing part, the complex antigen is dissociated from the antibody and the target to be detected in the specimen is captured, instead of the composite antibody, by the antibody, due to the difference in binding strength. By quantitatively determining the change due to the substitution reaction, the target can be quantitatively determined with a high sensitivity. For example, when the surface plasmon resonance measurement method is used, the change of the resonance angle θ due to the substitution reaction may be observed. The enhancement of the detection sensitivity due to the substitution method allows even the target having a concentration of ppt level to be detected.

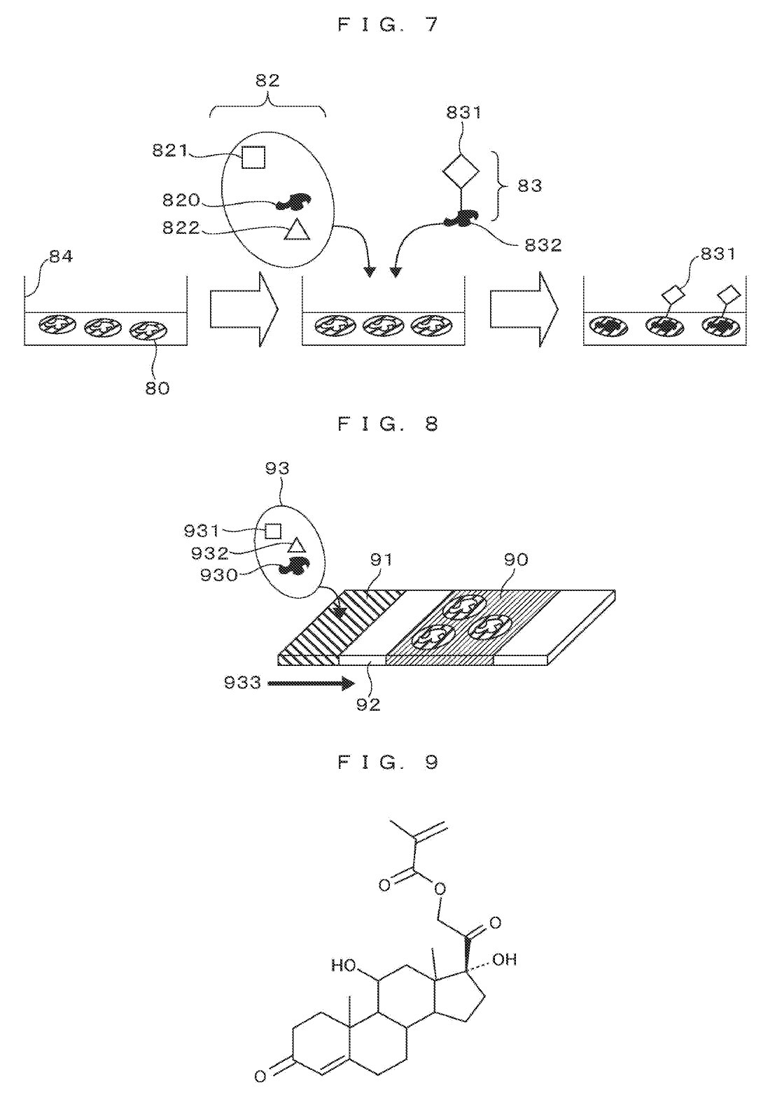

An example using the competition method is described on the basis of FIG. 7. As illustrated in FIG. 7, in a container 84, an aqueous suspension of a molecular-template polymer 80 is placed, and a solid or an aqueous solution of a specimen 82 and a labeled target 83 are placed in the container 84. The specimen 82 includes a target 820, a foreign substance A 821 and a foreign substance B 822. Needless to say, there possibly occurs a case in which the target 820 is absent, or a case in which a large number of types of foreign substances are present. The labeled target 83 is composed of a target moiety 832 and a label moiety 831. The target 820 and the labeled target 83 are allowed to react with the molecular-template polymer 80 for 1 hour at room temperature while the target 820 and the labeled target 83 are being allowed to compete against each other, and then the colorimetric magnitude or the fluorescence magnitude of the label moiety 831 is measured; and thus, the target amount in the specimen 82 can be derived. In other words, the larger the target amount, the smaller the colorimetric magnitude or the fluorescence magnitude. For the derivation of the target amount in the specimen 82, a separately derived colorimetric or fluorescence calibration line may be used. From this measurement, for example, cortisol contained in the specimen in a concentration of 125 μM or less can be quantitatively determined in a dominant manner.

Further, in the foregoing embodiment, the electric signal acquired in the part for measuring captured amount is frequently feeble in usual cases, and hence the acquired electric signal may be amplified if necessary. The amplification can be performed by means such as installation of an amplifier in the part for measuring captured amount. When the acquired electric signal is an analog signal, the analog signal may be subjected to AD conversion if necessary. The AD conversion can be performed by means such as installation of an AD converter such as a comparator in the part for measuring captured amount.

Furthermore, the part for measuring captured amount is constituted so as to be able to exhibit the measurement results. The destination of the measurement results is not particularly limited. For example, the measurement results may be exhibited on an external display device such as a monitor. When the measurement results are shown, the output form is not particularly limited. It may be an exhibition through direct wiring, or an exhibition through a cable with a provided connection terminal such as a USB terminal. Alternatively, the measurement results may also be wirelessly transmitted.

### THIRD EMBODIMENT

FIG. 8 shows a perspective view of an apparatus for detecting a chemical substance according to a third embodiment of the present invention. The apparatus for detecting a chemical substance of FIG. 8 is prepared by applying a molecular-template polymer to a material mainly such as a resin, glass, silica gel, paper or a metal. The detection apparatus is broadly composed of three parts, namely, a sample injection part 91, a capture/detection part 90, and a pretreatment layer 92. In the pretreatment layer 92, a non-woven fabric to adsorb proteins, lipids and the like in saliva is fixed. Accordingly, proteins, lipids and the like to disturb the detection of a steroid hormone such as cortisol are made not to enter the capture/detection part 90. The material used for the pretreatment layer 92 is not limited to a non-woven fabric, and may be a material such as a resin, glass, silica gel or paper. To the capture/detection part 90, the molecular-template polymer is applied. When the substitution method is utilized, a certain amount of a labeled target may be immobilized in advance. Next, a specimen 93 is applied to the sample injection part 91. The specimen 93 includes a target 930, a foreign substance A 931 and a foreign substance B 932. When the competition method is utilized, a labeled target is mixed in the specimen 93 and the resulting mixture is applied to the sample injection part 91. Subsequently, the specimen 93 and the labeled target travel in the direction of the arrow 933, and in the pretreatment layer 92, a fraction or the whole of the foreign substances in the specimen 93 is removed. Then, the target 930 and the labeled target in the specimen 93 are captured by the molecular-template polymer of the capture/detection part 90. By using a fluorescence microscope, visual verification, an optical microscope or the like for detection, the detection can be performed by evaluating the developed color or the like. By using this chip-shaped apparatus for detecting a chemical substance, for example, a target of a concentration of 50 μM or less in the specimen can be detected.

### EXAMPLES

Next, the present invention is described in more detail on the basis of Examples. However, following Examples are presented only for exemplification of the present invention, and the present invention is not limited by these Examples at all.

### Example 1

Descriptions are made on an example of the synthesis of molecular-template polymer particles utilizing the covalent bonds between a raw material and a target molecule, and target capturing test.

### (Methacroylation of Cortisol)

First, regarding the synthesis, cortisol as a template molecule was modified according to the following order to synthesize a cortisol derivative.

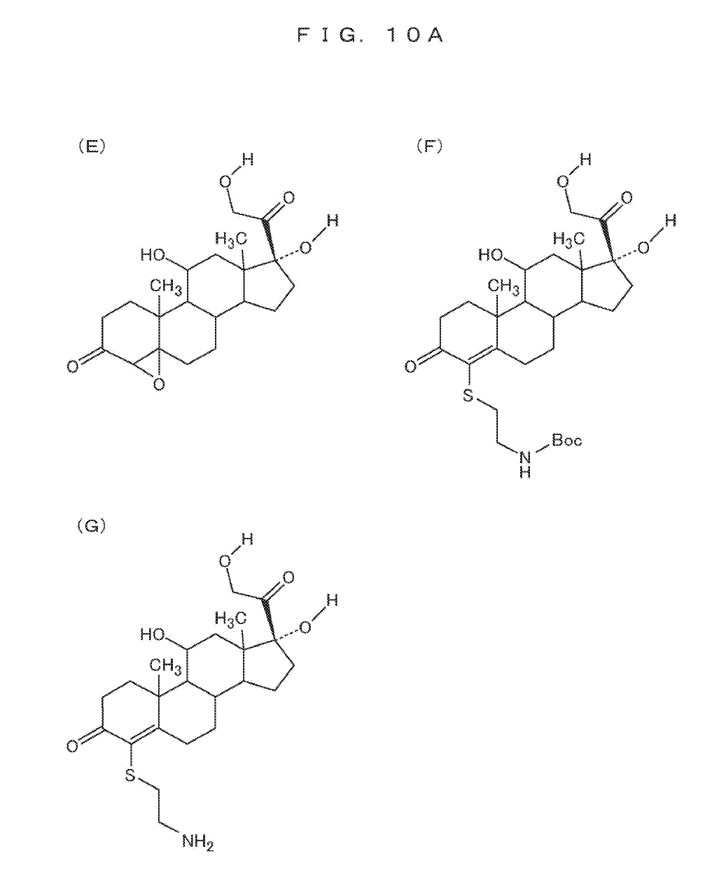

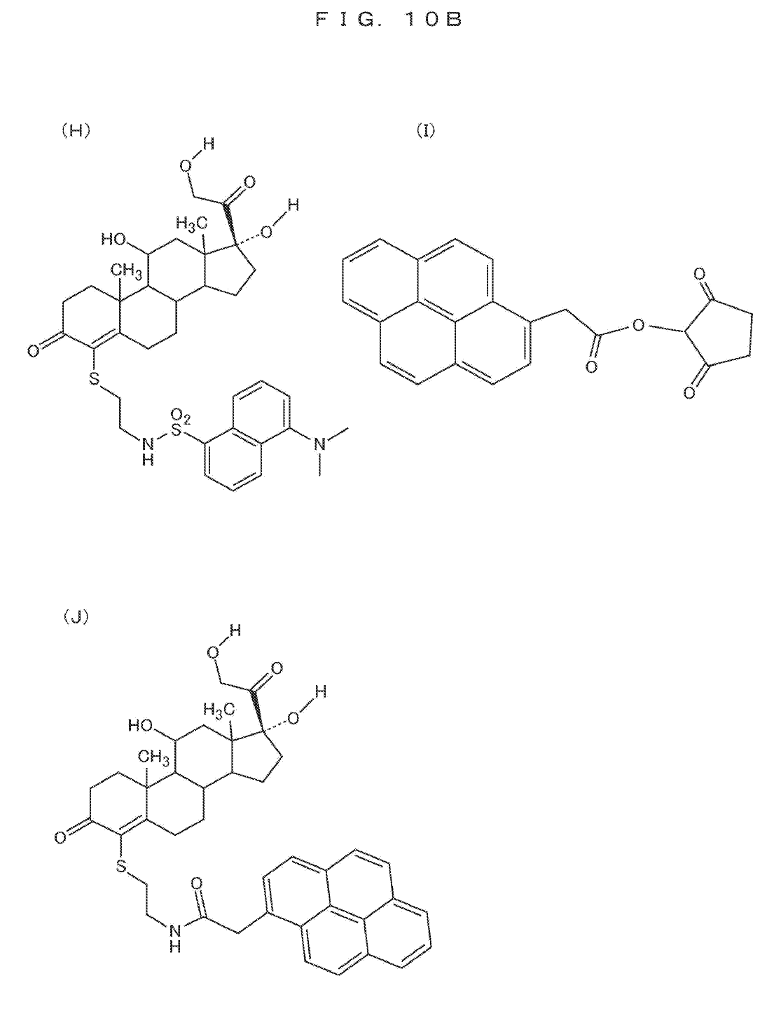

First, in nitrogen atmosphere, cortisol (2.5 mmol, 907 mg) was dissolved in dry THF (40 mL), and triethylamine (30 mmol, 4.2 ml) was added to the resulting solution and the solution was ice cooled. To this cooled solution, dry THF (40 mL) in which methacryloyl chloride (15 mmol, 1.5 ml) was dissolved was slowly dropwise added, and the solution was stirred at 0° C. for 1 hour, and then at room temperature for 4 hours. Subsequently, ethyl acetate was added to the reaction liquid, and the organic phase was washed with a saturated aqueous solution of sodium hydrogen carbonate, citric acid and an aqueous solution of sodium chloride by using a separating funnel. After that, the organic phase was dried over sodium sulfate. Next, the solvent was distilled off with an evaporator, and the extract was separated and purified with silica gel column chromatography (silica gel C-200, developing solvent:ethyl acetate/hexane=1:1) to yield a white solid (yield: 65%). FIG. 9 shows the molecular structure of methacroylated cortisol thus obtained.

Meanwhile, methacroylated cortisol illustrated in FIG. 9 can be also obtained by the following method. Specifically, in nitrogen atmosphere, in a two-neck flask, cortisol (2.5 mmol, 907 mg) and dimethylaminopyridine (0.25 mmol, 30.5 mg) were dissolved in dry THF (40 mL), and the resulting solution was ice cooled. Subsequently, triethylamine (30 mmol, 4.2 ml) and methacrylic anhydride (7.5 mmol, 1.2 ml) were slowly dropwise added to the solution, and the solution was stirred at 0° C. for 1 hour, and then at room temperature for 2 days. Ethyl acetate was added to the reaction liquid, and the organic phase was washed with pure water three times by using a separating funnel, and dried over sodium sulfate. The solvent was distilled off with an evaporator, and the extract was separated and purified with silica gel column chromatography (silica gel C-200, developing solvent:ethyl acetate/hexane=1:1) to yield a white solid (yield: 89%).

### (Synthesis of Particles as Core)

According to the recipe in Table 1, 760 mg (7.3 mmol) of styrene, 40 mg (0.31 mmol) of divinylbenzene (DVB), 79.2 g of water, and 41.3 mg (0.15 mmol) of V-50 (2,2′-Azobis(2-methylpropionamidine)dihydrochloride) were weighed in a two-neck flask. After replacing with nitrogen, the reaction was allowed to occur at 80° C. for 48 hours. The solution was then rapidly cooled in an ice bath and the reaction was terminated by injecting oxygen. During the reaction process, the reaction solution became white and turbid several hours after the reaction, and a white turbid emulsion was obtained after 48 hours. As a result of observation under an electron microscope, it was found that the particle diameter is 125 nm, exhibiting high uniformity of particle diameter.

TABLE 1
Raw material for synthesizing particles

| Raw material | Particles | |
|---|---|---|
| Styrene | 760 mg | (7.3 mmol) |
| DVB | 40 mg | (0.31 mmol) |
| Water | 79.2 g | |
| V-50 | 41.3 mg | (0.15 mmol) |

### (Synthesis of Molecular-Template Polymer Particles)

By using as a template molecule the cortisol derivative, having a methacryloyl group introduced thereto, prepared by any one of the foregoing methods, molecular-template polymer particles were synthesized according to the raw material composition shown in Table 2. A methacryloyl group has an ethylenically unsaturated group and is polymerization-reactive, and hence cortisol having a methacryloyl group introduced thereinto is copolymerizable with the added monomers shown in Table 2. Consequently, the raw material for the molecular-template polymer and the cortisol derivative can strongly bind to each other and recognize each other, and hence it is possible to produce a molecular-template polymer capable of capturing cortisol with a high selectivity.

Specifically, according to the recipe in Table 2, the polymerization of the Nano-MIP1 and Nano-MIP2 as a molecular-template polymer was performed.

### (Method for Synthesis of Nano-MIP1)

In a vial, the polystyrene suspension (3% by weight, 20 g/water) which has been synthesized according to Table 1 was added, and 3.9 mg (9 μmol) of methacroylated cortisol, 4.7 mg (36 μmol) of itaconic acid, and 69.0 mg (447.5 μmol) of methylene bisacrylamide were added and dissolved in suspension (THF). Then, after transfer into a φ18×180 mm test tube, 2.7 mg (9.85 μmol) of V-50 (2,2′-Azobis(2-methylpropionamidine)dihydrochloride), a polymerization initiator, was dissolved in the solution. The test tube was sealed with a septum cap and the air in the test tube was replaced with nitrogen, and then a polymerization reaction was allowed to occur at conditions including 80° C. and 800 rpm for 24 hours. The obtained polymerization liquid was recovered and added to a centrifuge to remove the supernatant. Then, it was subjected to hydrolysis for 24 hours using 50 ml of a 2 M aqueous solution of sodium hydroxide/methanol=1:1. After that, it was washed for several hours with 50 ml of 1 M hydrochloric acid/methanol=1:1 and 50 ml of pure water/methanol=1:1. According to this hydrolysis process and washing process, the cortisol derivative which has been introduced to the inside of the molecular-template polymer can be removed from the molecular-template polymer.

### (Method for Synthesis of Nano-MIP2)

In a vial, the polystyrene suspension (3% by weight, 20 g/water) which has been according to Table 1 was added, and 3.9 mg (9 μmol) of methacroylated cortisol, 4.7 mg (36 μmol) of itaconic acid, 59.5 mg (457 μWmol) of divinylbezene (DVB), and 9.5 mg (91.2 μmol) of styrene were added. Then, 3.2 mg (11.8 μmol) of V-50 (2,2′-Azobis(2-methylpropionamidine)dihydrochloride), a polymerization initiator, was dissolved in the solution. The test tube was sealed with a septum cap and the air in the test tube was replaced with nitrogen, and then a polymerization reaction was allowed to occur at conditions including 80° C. and 800 rpm for 24 hours. The obtained polymerization liquid was recovered and added to a centrifuge to remove the supernatant. Then, it was subjected to hydrolysis for 24 hours using 50 ml of a 2 M aqueous solution of sodium hydroxide/methanol=1:1. After that, it was washed for several hours with 50 ml of 1 M hydrochloric acid/methanol=1:1 and 50 ml of pure water/methanol=1:1. According to this hydrolysis process and washing process, the cortisol derivative which has been introduced to the inside of the molecular-template polymer can be removed from the molecular-template polymer. By following the aforementioned method, the core and shell type molecular-template polymer particles, which are molecular-template polymer particles for a steroid hormone and have a structure in which the molecular-template polymer consisting of a polymer capable of interacting with the steroid hormone coats the periphery of the particles, can be produced.

TABLE 2
Raw material for synthesizing two kinds
of molecular-template polymer particles

| Raw material | Nano-M1P1 | | Nano-M1P2 | |
|---|---|---|---|---|
| Suspension of | 20 g | | 20 g | |
| polystyrene particles | | | | |
| Methacroylated | 3.9 mg | (9 μmol) | 3.9 mg | (9 μmol) |
| cortisol | | | | |
| Itaconic acid | 4.7 mg | (36 μmol) | 4.7 mg | (36 μmol) |
| Methylene | 69.0 mg | (447.5 μmol) | None | |
| bisacrylamide | | | | |
| Styrene | None | | 9.5 mg | (91.2 μmol) |
| DVB | None | | 59.5 mg | (457 μmol) |
| V-50 | 2.7 mg | (9.85 μmol) | 3.2 mg | (11.8 μmol) |

### Example 2

### Fluorescent Labeling of Cortisol: Introduction of Dansyl Group

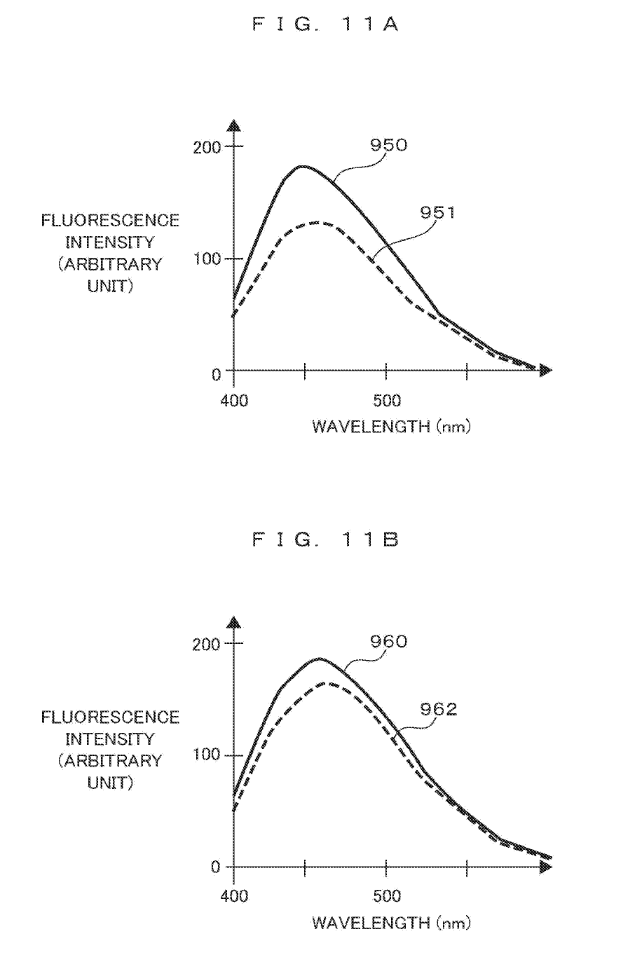

For detection of cortisol with high sensitivity, use of the fluorescent labeled cortisol was contemplated, and it was synthesized accordingly. The molecular structure of the molecule synthesized below is shown in (E) to (G) of FIG. 10A and (I) to (J) of FIG. 10B.

### Reaction (1): Epoxidation of Unsaturated Bond and Introduction of Amino Group

In a two-neck flask replaced with nitrogen, 1.82 g (5 mmol) of cortisol was weighed and partially dissolved in 65 ml of methanol and 25 ml of ethanol. After adjusting to 0° C. in an ice bath, 5 ml of 10% aqueous sodium hydroxide solution and 5 ml of 30% hydrogen peroxide (H₂O₂) were injected by using a syringe followed by reaction for 3 hours at 0° C. The reaction was further allowed to occur overnight at room temperature to obtain the cortisol derivative (E) as an intermediate. After that, 1 ml of 2-(Boc-amino)ethanethiol was added followed by the reaction at room temperature for 6 hours. The reaction solution was then neutralized with dilute hydrochloric acid. 30 ml of saturated brine was added. Extraction with ethyl acetate was performed three times and the organic phase was dried over sodium sulfate. The solvent was distilled off using an evaporator. The crude product was subjected to solvent fractionation using THF and chloroform, and the filtrate was separated and purified by column chromatography (developing layer: Silicagel C-200, developing solvent: chloroform/methanol/triethylamine=20/1/0.2). As a result, a yellowish white solid (cortisol derivative F) was obtained (yield: 20%).

### Reaction (2): Deprotection of Boc Group

To cortisol derivative F (54 mg, 0.1 mmol), 1 ml of 0.5 M hydrochloric acid/methanol solution was added. With shielding of sunlight, the reaction was allowed to occur for 4 hours at room temperature followed by neutralization with a saturated aqueous solution of sodium hydrogen carbonate. Saturated brine was added, and extraction with ethyl acetate was performed three times, and it was dried over sodium sulfate. As a result of distilling off the solvent, a yellowish brown solid (cortisol derivative G) was obtained (crude yield: 90%).

### Reaction (3): Dansylation

Under nitrogen atmosphere, dimethylaminopyridine (10 mg) was added to cortisol derivative G (30 mg, 0.069 mmol) and dissolved in 3 ml of distilled THF. After that, the mixture was dissolved in triethylamine (0.1 ml) and 2 ml of distilled THF. After adding dansyl chloride (20 mg, 1.1 eqv.) as a fluorescent molecule, the reaction was allowed to occur overnight at room temperature. The solvent was distilled off using an evaporator. Saturated brine was added, and extraction with dichloromethane was performed three times, and the organic phase was dried over sodium sulfate. As a result of removing the solvent by distillation, a viscous yellow solid was obtained. The crude product was dissolved in THF, and as a result of performing separation and purification by fractional TLC (developing layer: Silicagel C-200, developing solvent: chloroform/methanol/triethylamine=20/1/0.2), a yellowish white solid was obtained (cortisol derivative H).

### (Fluorescence Measurement of Fluorescent Labeled Cortisol Derivative (H))

The fluorescent labeled cortisol derivative (H) was dissolved in chloroform and the fluorescence spectrum was measured by using a fluorescence spectrophotometer. As a result of determining the fluorescence spectrum when excited at excitation wavelength (375 nm), the maximum fluorescence peak was found near 450 nm. As such, by using the fluorescent labeled cortisol derivative (H), the detection power of the molecular-template polymer particles (Nano-MIP1, Nano-MIP2) for cortisol was evaluated.

### [Cortisol Detection Test]

The cortisol adsorption power of the Nano-MIP1 and Nano-MIP2, which have been produced according to the aforementioned method, was evaluated. A suspension of Nano-MIP1 and Nano-MIP2 was centrifuged followed by removal of a solvent and substituted three times with chloroform/hexane=4/1. Because the Nano-MIP1 was aggregated in chloroform/hexane=4/1, it was not possible to carry out the fluorescence measurement. Thus, only the Nano-MIP2 was used for the following titration test.

3 ml of 50 μM solution of the fluorescent labeled cortisol derivative (H) (chloroform/hexane=4/1) was weighed in a fluorescence cell. Under stirring, the Nano-MIP2 was added, in an amount of 100 μl for every 10 minutes, and the fluorescence was measured at excitation wavelength (375 nm). The dropwise addition and measurement were repeated until the dropwise addition amount is 400 μl. As a reference example, only the solvent (chloroform/hexane=4/1) (100 μl) was added dropwise, and the fluorescence was measured at excitation wavelength (375 nm).

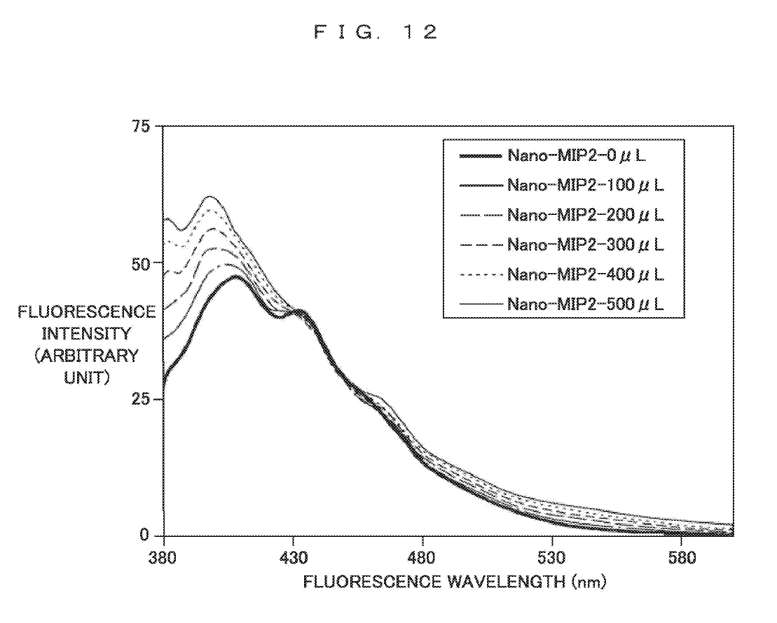

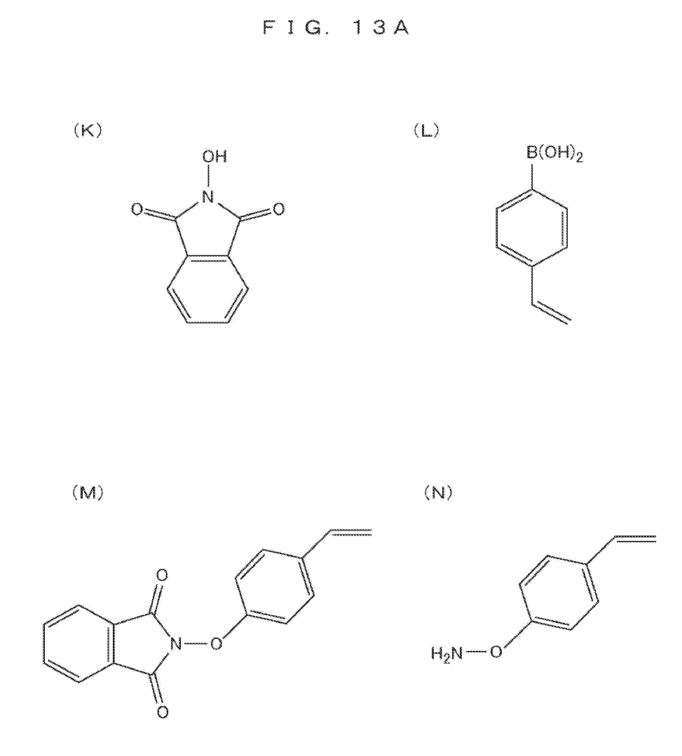

As a result, when the suspension of Nano-MIP2 was added dropwise, the wavelength having maximum fluorescence intensity was shifted to a long wavelength side and the fluorescent intensity was greatly decreased compared to a case in which only the solvent was added. The results are shown in FIG. 11A, FIG. 11B and Table 3. In the graph of FIG. 11A, the horizontal axis represents the wavelength (nm) and the vertical axis represents the fluorescence intensity (arbitrary unit). The solid line 950 of the graph of FIG. 11A is a spectrum before addition of molecular-template polymer particles. The broken line 951 is a spectrum after addition of 400 μl of molecular-template polymer particles. The fluorescence intensity (arbitrary unit) at wavelength of 450 nm was shown.

Furthermore, in the graph of FIG. 11B, the horizontal axis represents the wavelength (nm) and the vertical axis represents the fluorescence intensity (arbitrary unit). The solid line 960 of the graph of FIG. 11B is a spectrum before addition of molecular-template polymer particles. The broken line 962 is a spectrum after addition of 400 μl of a solvent only. The fluorescence intensity (arbitrary unit) at wavelength of 450 nm was shown.

In Table 3, a change in the fluorescence intensity (arbitrary unit) caused by the added solution described above is shown. When the addition amount was 0 μl (before addition), the fluorescence intensity was 180 for both cases of adding Nano-MIP2 and adding a solvent only. However, according to addition of the Nano-MIP2, the fluorescence intensity was 160 at the addition amount of 100 μl. The fluorescence intensity was 150 at the addition amount of 200 μl. The fluorescence intensity was 135 at the addition amount of 300 μl. The fluorescence intensity was greatly decreased to 125 at the addition amount of 400 μl.

Meanwhile, when only the solvent was added, the fluorescence intensity was 175 at the addition amount of 100 μl. The fluorescence intensity was 170 at the addition amount of 200 μl. The fluorescence intensity was 165 at the addition amount of 300 μl. The fluorescence intensity was decreased to 160 at the addition amount of 400 μl.

From the above results, it was found that, compared to simple dilution in which only the solvent is added, the fluorescence intensity is greatly decreased if the Nano-MIP2 is added. As such, it is believed that the fluorescent labeled cortisol is introduced to the inside of the molecular-template polymer particles, and as a result of interaction among the molecules, the fluorescence intensity was greatly decreased. Since the concentration of the fluorescent labeled cortisol was 50 μM for this case, according to the present invention, at least 50 μM of cortisol can be detected. Meanwhile, although this detection method is based on direct measurement of fluorescence intensity, a competition method or a substitution method described above can be used.

TABLE 3
Change in fluorescence intensity caused
by added solution (wavelength: 450 nm)

Fluorescence intensity
(arbitrary unit)

| Addition | Addition of | Addition of |
|---|---|---|
| amount | Nano-M1P2 | solvent only |
| 0 μl (Before | 180 | 180 |
| addition) | | |
| 100 μl | 160 | 175 |
| 200 μl | 150 | 170 |
| 300 μl | 135 | 165 |
| 400 μl | 125 | 160 |

### Example 3

### Fluorescent Labeling of Cortisol

### Introduction of Pyrene

For detection of cortisol with high sensitivity, use of the fluorescent labeled cortisol was contemplated, and it was synthesized accordingly. In the above, a detection example using cortisol introduced with a dansyl group was described. Subsequently, a detection example using cortisol introduced with pyrene is described.

### Reaction (5) Synthesis of Pyrene Active Ester

Under nitrogen atmosphere, 1-pyrene Acetic Acid (260.3 mg, 1 mmol) was dissolved in distilled THF (5 mL). To the solution, 1-(3-dimethylaminopropyl)-3-ethylcarbodiimide (EDC) (212 μl, 1.2 mL) diluted with distilled THF (1 ml) and N-hydroxy succinimide (138.1 mg, 1.2 mmol) dissolved in distilled THF (5 ml) were added and the mixture was stirred overnight at room temperature while shielding sunlight. Upon the completion of the reaction, the reaction solution was distilled off using an evaporator and extraction with methylene chloride was performed three times after adding pure water. The organic phase was dried over sodium sulfate, and as a result of distilling off the solvent by an evaporator, a brownish-red solid was obtained. The obtained solid was subjected to decantation using ethyl acetate, and the supernatant was subjected to separation and purification based on column chromatography (developing layer: C-200, developing solvent: ethyl acetate/hexane=1:1) to yield a yellow solid (pyrene derivative, molecular structure I of FIG. 10B) (yield: 83%).

### Reaction (6) Synthesis of Pyrene-Labeled Cortisol

Under nitrogen atmosphere, the cortisol derivative G (61 mg, 0.14 mmol) was dissolved in methylene chloride (3 ml) and N,N-dimethyl-4-aminopyridine (DMAP) (17.2 mg, 0.14 mmol) dissolved in methylene chloride (1 ml) was added thereto. Subsequently, the pyrene derivative (I: synthesized in the aforementioned Section 1.) (50 mg, 0.14 mmol) dissolved in methylene chloride (3 ml) was added, and the reaction was allowed to occur overnight at room temperature while shielding sunlight. Upon the completion of the reaction, extraction with methylene chloride was performed three times after adding pure water. The organic phase was dried over sodium sulfate, and as a result of distilling off the solvent by an evaporator, a viscous brownish-red solid was obtained. The obtained solid was subjected to decantation using ethyl acetate, and the supernatant was subjected to separation and purification based on column chromatography (developing layer: C-200, developing solvent: ethyl acetate/hexane=1:4) to yield a yellow solid (molecular structure J of FIG. 10B) (yield: 64%).

### [Cortisol Detection Test]

The cortisol (J) introduced with pyrene as synthesized above was dissolved in chloroform and the fluorescence spectrum was measured at excitation wavelength (350 nm). As a result, the peak with maximum fluorescence was determined near 400 nm.

Subsequently, the interaction with a molecular-template polymer was determined. A solution (chloroform/hexane=4/1) of the derivative (J) at concentration of 1 μmol/l was prepared, and 3 ml of the solution was weighed in a fluorescence cell. Then, under stirring, the Nano-MIP2 was dropwise added, in an amount of 0, 100, 200, 300, 400, or 500 μl for every 10 minutes, and the measurement was performed at excitation wavelength (350 nm). Meanwhile, a suspension of the Nano-MIP2 polymer was used after it was prepared to have a solid matter concentration of about 1 mg/mL.

The obtained fluorescence spectrum is shown in FIG. 12. In the graph of FIG. 12, the horizontal axis represents the wavelength (nm) and the vertical axis represents the fluorescence intensity (arbitrary unit). The result before the addition (0 μl) is shown with a grey solid line. Thereafter, by adding a suspension of the Nano-MIP2 polymer in an amount of 100 μl, the fluorescence intensity has increased at every wavelength as shown by the black long-dashed line. Further, the wavelength at which a peak with the maximum fluorescence intensity is shown was shifted to a short wavelength side in the wavelength range of 380 to 600 nm at the time. After that, after further addition of a suspension of the Nano-MIP2 polymer in an amount of 100 μl (total addition amount of 200 μl), the fluorescence spectrum was shown with a grey broken line. As a result, the wavelength at which a peak with the maximum fluorescence intensity is shown was shifted to a short wavelength side like the above. Then, like the above, after further addition of a suspension of the Nano-MIP2 polymer in an amount of 100 μl (total addition amount of 300 μl), the fluorescence spectrum was shown with a black medium-dashed line. Then, like the above, after further addition of a suspension of the Nano-MIP2 polymer in an amount of 100 μl (total addition amount of 400 μl), the fluorescence spectrum was shown with a grey fine-dashed line. Then, like the above, after further addition of a suspension of the Nano-MIP2 polymer in an amount of 100 μl (total addition amount of 500 μl), the fluorescence spectrum was shown with a black solid line.

Among the fluorescence spectrums obtained above, within the wavelength range of 380 nm to 600 nm, the wavelength at which the maximum fluorescence intensity is shown after adding a suspension of the Nano-MIP2 polymer in each amount and the fluorescence intensity are shown in Table 4.

TABLE 4
Wavelength having maximum fluorescence intensity after addition
of a suspension of the Nano-MIP2 polymer in each addition amount
and fluorescent intensity (wavelength range: 380 nm to 600 nm)

| Total addition | Wavelength | Fluorescence intensity |
|---|---|---|
| amount | (nm) | (arbitrary unit) |
| 0 μL (Before | 409.5 | 48.33 |
| addition) | | |
| 100 μl | 404.0 | 49.11 |
| 200 μl | 401.0 | 52.29 |
| 300 μl | 399.0 | 55.67 |
| 400 μl | 402.0 | 60.15 |
| 500 μl | 398.0 | 61.61 |

From the above results, it was determined that 1 μmol/l cortisol can be detected by the MIP of the present invention as seen from the change in fluorescence spectrum. It was also confirmed that the detection can be similarly made for 10 μmol/l cortisol.

### Example 4

Descriptions are made for examples of synthesis of molecular-template polymer particles utilizing the covalent bond at two points between a raw material and a target molecule, and a target capturing test.

### (Synthesis of Cortisol Derivative Having Two Polymerizable Substituent Groups)

### Reaction (7): Synthesis of Synthetic Intermediate

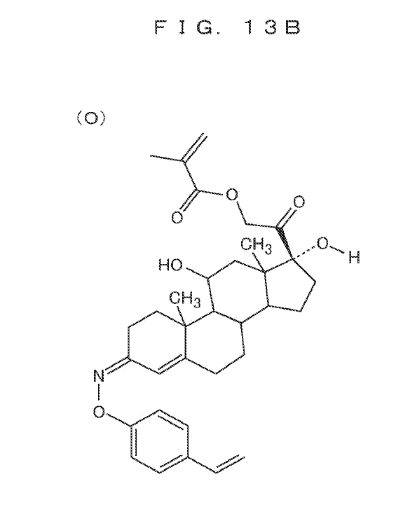

To a 50 mL branched flask, N-hydroxylphthalimide (molecular structure K of FIG. 13A) (163 mg, 1 mmol), CuCl (I) (99 mg, 1 mmol), activated molecular sieves 4 A (200 mg), 4-vinylphenyl boronic acid (molecular structure L of FIG. 13A) (296 mg, 2 mmol), and a stirrer bar were added, and after further adding 1,2-dichloroethane (5 mL) thereto, the mixture was dissolved and suspended. As for the molecular sieves 4 A, those obtained after activation overnight under vacuum at 150° C. were used. Pyridine (90 μL) was added thereto and stirred. As a result, a brown suspension was yielded. After that, the reaction solution was turned into green color. Upon the completion of the reaction, the reaction solution was adsorbed onto a silica gel, the solvent was directly distilled off under reduced pressure, and each spot was eluted with ethyl acetate. After that, by using an auto column, an attempt was made to separate the synthetic intermediate molecule M (FIG. 13A). The conditions for separation are as follows. Only hexane was allowed to flow for 12 minutes, and gradient flow was allowed to flow for 11 minutes to have finally hexane:ethyl acetate=9:1. After that, the liquid was allowed to flow for 20 minutes with the ratio of 9:1. The amount of the obtained synthetic intermediate molecule M was 137 mg (0.51 mmol) with yield of 52%.

### Reaction (8): Synthesis of Functional Monomer (Molecule N)

To a 50 mL branched flask, the synthetic intermediate molecule M (82.6 mg, 0.324 mmol), CHCl₃ (5 mL) prepared to have 10% MeOH, and hydrazine monohydrate (47.5 μL, 0.972 mmol) were added and stirred overnight at room temperature condition. White precipitates were precipitated immediately after starting the reaction. Stirring was performed overnight. Thereafter, the precipitates were directly adsorbed onto silica gel and washed by flowing a hexane solution of 30% ethyl acetate through 5 g of silica gel. Unreacted hydrazine was removed at that time. The residuals containing the functional monomer (molecule N) were used for the next step as they remain in a crude state.

### Reaction (9): Synthesis of Cortisol Derivative Having Two Polymerizable Substituent Groups

After the synthesis, the functional monomer (molecule N) was used for the following reaction as they remain in a crude state. The solvent contained in the solution in a crude state was distilled off under reduced pressure, and the mixture having the functional monomer (molecule N) (0.63 mmol, injection amount of the synthesis intermediate of reaction (8)), methacryloylated cortisol (167.2 mg, 0.342 mol), and NaOAc (0.68 mmol) were dissolved in 10 mL of MeOH and the reaction was allowed to occur for 48 hours at room temperature while shielding sunlight. Upon the completion of the reaction, the reaction solution turned into brownish-red color. After that, the solvent was distilled off under reduced pressure, and by adding CH₂Cl₂, NaOAc was precipitated and filtered. The solution was then separated by using an auto column. The separated solution was distilled off under reduced pressure, and subjected to identification by using ¹H-NMR and MALDI-TOF-MS. As a result, the molecule 0 was obtained with an amount of 8 mg and the yield was 4%.

By using the disubstituted cortisol derivative which has been synthesized above, a molecular-template polymer was synthesized according to the method of Example 1 to Example 3. Furthermore, by using the labeled cortisol, detection of cortisol was carried out. From the change in fluorescence spectrum, it was confirmed that the cortisol at 1 μmol/L could be detected by using MIP based on Example 4. It was also confirmed that the cortisol at 10 μmol/L could be similarly detected.

As presented above, the embodiments for implementing the present invention are described. The molecular-template polymer has selectivity and a capturing property like those of an antibody as a biopolymer, and it is excellent in environmental tolerance and temperature resistance, because of being a non-natural synthetic substance. Accordingly, it has an advantage that users can use MIP, for example, without being bothered about the storage. Consequently, it is possible to provide a chemical sensor easy to use for general consumers at home as well as medical personnel (medical doctors, medical technicians and nurses), assumed as users. In particular, since a steroid hormone such as cortisol which is closely related to stress disorder can be detected with high sensitivity, it is useful for early diagnosis of the symptoms of stress disorder, and thus can contribute to the prevention and early treatment of stress disorder.

The present invention should not be limited to the foregoing embodiments and may include various modified examples. For example, the constitution of some embodiment may be partially replaced with another embodiment, and the constitution of some embodiment may include another embodiment as added thereto. Alternatively, the constitutions of the respective embodiments may be partially modified by adding or deleting other constitutions, or by replacing with other constitutions.

### REFERENCE SINGS LIST

1 Apparatus for detecting a chemical substance
10 Molecule capturing part
101 Capturing body
102 Support
103 Molecular-template polymer
104 Molecular-template polymer
11 Part for measuring captured amount
111 Arrow
112 Arrow
14 Sample injection part
15 Sample conveying part
16 Discharge part
17 Specimen
170 Target
171 Foreign substance A
172 Foreign substance B
20 Target
201 Monomer raw material A
202 Monomer raw material B
203 Monomer raw material C
21 Recognition site
22 Molecular-template polymer
25 Particles
26 Molecular-template polymer
261 Recognition site
27 Particles
28 Molecular-template polymer
501 Dotted line
502 Dotted line
6 Sample chamber
7 Liquid flow path part
8 Flow injection port
80 Molecular-template polymer
82 Specimen
83 Labeled target
820 Target
821 Foreign substance A
822 Foreign substance B
832 Target moiety
831 Label moiety
84 Container
9 Flow discharge port 1
90 Capture/detection part
91 Sample injection part
92 Pretreatment layer
93 Specimen
930 Target
931 Foreign substance A
932 Foreign substance B
933 Arrow
950 Solid line
951 Broken line
960 Solid line
961 Broken line

The publications, patents and patent applications quoted in present specification are all incorporated in the present specification by reference.

1.-25. (canceled)

26. Molecular-template polymer particles for cortisol or a derivative thereof in which the polymer is coated with polystyrene and the polymer comprises itaconic acid.

27. The molecular-template polymer particles according to claim 26,
wherein the molecular-template polymer particles comprising a polymer that interacts with a steroid hormone, and
wherein the polymerization unit of the polymer has at least two functional groups that interact with the steroid hormone.

28. The molecular-template polymer particles according to claim 26, wherein the polymerization unit of the polymer has at least two carboxylic acid groups as a functional group that interacts with the steroid hormone.

29. A chemical substance detection apparatus comprising:
a molecule capturing part which has a capturing body containing the molecular-template polymer particles according to claim 26,
wherein the molecular-template polymer particles comprising a polymer that interacts with a steroid hormone,
the chemical substance detection apparatus comprising:
a part for measuring captured amount for quantifying the steroid hormone captured in the molecule capturing part.

30. The chemical substance detection apparatus according to claim 29,
wherein the molecule capturing part is configured to enhance the detection sensitivity for the steroid hormone based on a competition method or a substitution method.

31. The chemical substance detection apparatus according to claim 29, wherein the steroid hormone is quantified in the part for measuring captured amount by using a surface plasmon resonance measurement method, a quartz crystal microbalance measurement method, an electrochemical impedance method, a colorimetric method, or a fluorescence method.

32. A method for detecting a chemical substance, the method comprising steps of:
capturing a steroid hormone on a molecule capturing part by contacting the molecule capturing part, which has a capturing body comprising the molecular-template polymer particles according to claim 26, with a specimen containing the steroid hormone; wherein the molecular-template polymer particles comprising a polymer that interacts with the steroid hormone, and
quantifying the steroid hormone captured by the molecule capturing part.

33. The method for detecting a chemical substance according to claim 32, wherein the molecule capturing part is configured to enhance the detection sensitivity for the steroid hormone based on a competition method or a substitution method.

34. A cortisol derivative introduced with a fluorescent molecule in which the fluorescent molecule is linked to the position 4 of the cortisol via a sulfur atom.
